(19) United States
(12) Patent Application Publication
(10) Pub. No.: US 2004/0038886 A1
(43) Pub. Date: Feb. 26, 2004

(54) Chimeric cytoplasmic signalling molecules derived from cd137

(76) Inventors: Helene Margaret Finney, Berkshire (GB); Alastair David Griffiths Lawson, Hampshire (GB)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/399,364

(22) PCT Filed: Oct. 16, 2001
(86) PCT No.: PCT/GB01/04611

(30) Foreign Application Priority Data
Oct. 16, 2000 (GB) 0025307.0

(51) Int. Cl. A61K 38/17; C07K 14/47; C12P 21/02; C12N 5/06

(52) U.S. Cl. 514/12; 536/23.5; 530/350; 435/69.1; 435/320.1; 435/325

(57) ABSTRACT

Nucleic acids are described which code for chimeric cytoplasmic signalling molecules containing at least one cytoplasmic signalling sequence derived from CD137. The nucleic acids may be expressed in cells to produce chimeric receptors and other proteins which are able to regulate cell activation processes with improved efficiency. Such regulated cells are of use in medicine, for example in the treatment of infectious, inflammatory and autoimmune diseases.

The present invention relates to novel cytoplasmic signalling molecules, the nucleic acids encoding them and the use of these nucleic acids and cytoplasmic signalling molecules in medicine and research.

Throughout this application various publications are referenced by author and year of publication. Full citations for these publications are provided following the detailed description of the invention and examples.

CD137, which is also known as 4-1BB or ILA, is a member of the TNF-receptor (TNFR) superfamily (Kwon & Weissman, 1989) and as such, shares a number of structural and functional characteristics of the superfamily. The extracellular domain of CD137 contains four characteristic cysteine-rich TNFR motifs and signal transduction through CD137 is mediated by the association of the tumour necrosis factor receptor-associated factors (TRAFs) TRAF1, TRAF2 and TRAF3 with the cytoplasmic domain of CD137. This is thought to result in downstream signal transduction through several pathways, including the c-Jun N-terminal kinase (JNK)/stress-activated kinase (SAPK) pathways. As with other TNFR superfamily members, CD137 has been shown to participate as a co-stimulatory molecule in T cell activation (Goodwin et al., 1993, DeBenedette et al., 1995, 1997), although it is becoming clear that this is not the only role of the molecule, there is an increasing body of evidence to suggest that CD137, like other TNFR superfamily members, plays many diverse roles in the immune response (Kwon et al., 2000), following T-cell activation.

Research in the area of immune cell signalling has yielded a considerable amount of information about the signal transduction events that occur downstream of antigen receptor engagement. A substantial number of studies have concentrated on the receptors themselves, and the enzymes stimulated in response to antigen binding (reviewed by Weiss & Littman, 1994; DeFranco, 1997).

Individual components of the T cell receptor (TCR) complex have been well characterised and in a number of cases the functionality of receptor sub-units or domains has been determined though the construction of chimeric receptor proteins (Kuwana et al., 1987; Romeo et al., 1992). Cytoplasmic signalling domains in particular, and their role in TCR activation, have been identified using this approach. This information has led to the development of chimeric receptors that are capable of regulating cell activation processes (see for example Finney et al., 1998 and published International Patent Specifications WO 97/23613, WO 96/23814, WO 96/24671, WO 99/00494, WO 99/57268).

The ability to control the biological effects of cellular activation, for example, increased cellular proliferation, increased expression of cytokines, stimulation of cytolytic activity, differentiation of other effector functions, antibody secretion, phagocytosis, tumour infiltration and/or increased cellular adhesion, with chimeric receptors has considerable therapeutic potential.

Whilst currently available chimeric receptors are capable of effectively activating cells, there is room for improvement in the efficacy with which the cytoplasmic signalling domain of such a chimeric receptor transduces the signal from the extracellular ligand binding domain to downstream members of secondary messenger pathways, typically the src and syk-tyrosine kinase family.

The current invention addresses these difficulties by providing novel cytoplasmic signalling molecules that comprise at least part of the CD137 polypeptide and are thus capable of engaging an alternative signal transduction pathway to that used by the cytoplasmic signalling domains of previously described chimeric receptors. The current invention also provides novel cytoplasmic signalling molecules capable of mediating cellular activation more efficiently by transducing signals through more than one secondary messenger pathway.

We have been able to demonstrate that the use of the CD137 polypeptide as part of a chimeric receptor, results in cellular activation in response to extracellular ligand binding to the receptor. In particular, where CD137 is employed in conjunction with at least one other cytoplasmic signalling sequence to form a cytoplasmic signalling molecule, this can confer novel and unexpected properties on the cytoplasmic signalling molecule. For example, where a cytoplasmic signalling molecule comprising CD137 in conjunction with two further cytoplasmic signalling sequences is employed as the cytoplasmic signalling domain of a chimeric receptor, the resulting levels of cellular activation are much higher than would be predicted. Most surprisingly, the observed degree of activation exceeds that produced via any previously constructed chimeric receptor by a considerable margin.

Thus, according to the present invention, there is provided a nucleic acid encoding a cytoplasmic signalling molecule comprising two cytoplasmic signalling sequences, wherein each cytoplasmic signalling sequence mediates signal transduction through a different secondary messenger pathway. Preferably, a cytoplasmic signalling molecule of the invention will transduce signals via tumour necrosis factor receptor-associated factors (particularly, for example TRAF2), to pathways such as the c-Jun N-terminal kinase (JNK)/stress-activated kinase (SAPK) pathways. More preferably, such a cytoplasmic signalling molecule will also mediate signal transduction via the src or syk-tyrosine kinase pathways.

In one particular embodiment, the invention provides a nucleic acid encoding a cytoplasmic signalling molecule comprising at least two cytoplasmic signalling sequences, wherein at least one cytoplasmic signalling sequence is derived from CD137.

As described above, a cytoplasmic signalling molecule of the invention comprises more than one cytoplasmic signalling sequence. The term “cytoplasmic signalling sequence” as used herein, means any sequence of amino acids that form at least a substantial part of a larger domain and are known to function as a unit capable of transducing a signal, which results in the activation or inhibition of biological processes within a cell.

The CD137 polypeptide consists of an extracellular domain, which contains three TNFR repeats, a transmembrane domain, and a cytoplasmic domain that is responsible for intracellular signal transduction. Accordingly a cytoplasmic signalling sequence derived from CD137 will comprise the cytoplasmic domain of CD137, or a derivative or variant thereof that has the same functional capability. By the term “variant” is meant any species variant, or any variant comprising one or more amino acid substitution, deletion or addition, provided that the variant retains the same functional capability as original parent sequence. Preferably cytoplasmic signalling molecules of the invention will contain a cytoplasmic signalling sequence comprising amino acids residues 214 to 255 of CD137 (Alderson et al., 1994), or a derivative or variant thereof.

Preferred examples of further cytoplasmic signalling sequences for use in the invention include the cytoplasmic sequences of the TCR and co-receptors that act in concert to initiate signal transduction following antigen receptor engagement, as well as any derivative or variant (as described above) of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signalling sequence: those that initiate antigen-dependent primary activation through the TCR (primary cytoplasmic signalling sequences) and those that act in an antigen-independent manner to provide a secondary or co-stimulatory signal (secondary cytoplasmic signalling sequences).

In one aspect of the invention a cytoplasmic signalling molecule will comprise a cytoplasmic signalling sequence derived from CD137 and a primary cytoplasmic signalling sequence.

Primary cytoplasmic signalling sequences regulate primary activation of the TCR complex either in a stimulatory way, or in an inhibitory way. Primary cytoplasmic signalling sequences that act in a stimulatory manner may contain signalling motifs which are known as immunoreceptor tyrosine-based activation motifs or ITAMs (Reth, 1989), whereas those that act in an inhibitory manner may contain signalling motifs known as immunoreceptor tyrosine-base inhibition motifs or ITIMs (Burshtyn et al., 1999).

Thus primary cytoplasmic signalling sequences for use in this or any aspect of the invention described herein, will preferably contain either an immunoreceptor tyrosine-based activation motif (ITAM), or an immunoreceptor tyrosine-based activation motif (ITIM).

Examples of ITAM containing primary cytoplasmic signalling sequences that are of particular use in the invention include those derived from TCRζ, FcRγ, FcRβ, CD3γ, CD3δ, CD3ε, CD5, CD22, CD79a, CD79b, and CD66d. Preferably cytoplasmic signalling sequences derived from these molecules will comprise amino acid residues 31-142 of TCRζ, amino acids residues 27-68 of FcRγ, amino acid residues 201-244 of FcRβ, amino acid residues 117-160 of CD3γ, amino acid residues 107-150 of CD3δ, amino acid residues 378-471 of CD5, amino acid residues 688-828 of CD22, amino acid residues 134-194 of CD79a, amino acid residues 154-201 of CD79b, or residues 143-218 of CD66d.

It is particularly preferred that cytoplasmic signalling molecules according to this aspect of the invention comprises a cytoplasmic signalling sequence derived from TCRζ.

In an alternative embodiment, a cytoplasmic signalling molecule of the invention will comprise a cytoplasmic signalling sequence derived from CD137 and a secondary cytoplasmic signalling sequence.

Molecules containing secondary cytoplasmic signalling sequences suitable for use in this or any aspect of the invention described herein include CD2, CD4, CD5, CD8α, CD8β, CD28, CD134, and CD154. Preferably cytoplasmic signalling sequences derived from these molecules will comprise amino acid residues 212-327 of CD2, amino acid residues 396-433 of CD4, amino acid residues 186-214 of CD8α, amino acid residues 175-189 of CD8β, amino acid residues 162-202 of CD28, amino acid residues 213-249 of CD134, or amino acid residues 1-22 of CD154. It is particularly preferred that secondary signalling sequences derived from CD28 or CD154 are employed in conjunction with a cytoplasmic signalling sequence derived from CD137.

As described above, we have found that a cytoplasmic signalling molecule comprising CD137 in conjunction with two further cytoplasmic signalling sequences is particularly efficient at mediating signal transduction when employed as the cytoplasmic signalling domain of a chimeric receptor.

A further aspect of the invention therefore provides a nucleic acid encoding a cytoplasmic signalling molecule comprising a cytoplasmic signalling sequence derived from CD137 and at least two further cytoplasmic signalling sequences.

One or both of the at least two additional cytoplasmic signalling sequences may be either a primary cytoplasmic signalling sequence or a secondary cytoplasmic signalling sequence as described hereinbefore. However, it is especially preferred that at least one additional cytoplasmic signalling sequence will be a primary cytoplasmic sequence and at least one other cytoplasmic signalling sequence will be a secondary cytoplasmic signalling sequence. Whilst any of the primary and secondary cytoplasmic signalling sequences described above may be incorporated into a cytoplasmic signalling molecule according to this aspect of the invention, the combination of a primary signalling sequence derived from TCRζ with a secondary signalling sequence derived from CD28 is preferred.

The cytoplasmic signalling sequences within a cytoplasmic signalling molecule of the invention may be linked to each other in a random or specified order. Optionally, a short oligo- or polypeptide linker, preferably between 2 and 10 amino acids in length may form the linkage. A glycine-serine doublet provides a particularly suitable linker.

Nucleic acids encoding cytoplasmic signalling molecules comprising cytoplasmic CD137 and one additional cytoplasmic signalling sequence may thus encode in reading frame: i) a cytoplasmic signalling sequence derived from CD137 and ii) a primary cytoplasmic signalling sequence; i) a primary cytoplasmic signalling sequence and ii) a cytoplasmic signalling sequence derived from CD137; i) a cytoplasmic signalling sequence derived from CD137 and ii) a secondary cytoplasmic signalling sequence; or i) a secondary cytoplasmic signalling sequence and ii) a cytoplasmic signalling sequence derived from CD137. Specific examples of such cytoplasmic signalling molecules include those that comprise, in order from the amino to carboxyl terminus, cytoplasmic signalling sequences derived from i) CD137 and ii) TCRζ; i) TCRζ and ii) CD137; i) CD137 and ii) CD28; and i) CD28 and ii) CD137.

Where cytoplasmic signalling molecules of the invention comprise a cytoplasmic signalling sequence derived from CD137 and at least two additional cytoplasmic signalling sequences, these may also be linked in random or specified order, optionally via a linker as described above. Thus nucleic acids encoding such cytoplasmic signalling molecules may encode i) a cytoplasmic signalling sequence derived from CD137, ii) a primary cytoplasmic signalling sequence and iii) a secondary cytoplasmic signalling sequence; i) a cytoplasmic signalling sequence derived from CD137, ii) a secondary cytoplasmic signalling sequence and iii) a primary cytoplasmic signalling sequence; i) a primary cytoplasmic signalling sequence, ii) a cytoplasmic signalling sequence derived from CD137 and iii) a secondary cytoplasmic signalling sequence; i) a primary cytoplasmic signalling sequence, ii) a secondary cytoplasmic signalling sequence and iii) a cytoplasmic signalling sequence derived from CD137; i) a secondary cytoplasmic signalling sequence ii) a primary cytoplasmic signalling sequence and iii) a cytoplasmic signalling sequence derived from CD137; or i) a secondary cytoplasmic signalling sequence, ii) a cytoplasmic signalling sequence derived from CD137 and iii) a primary cytoplasmic signalling sequence.

Specific examples of such cytoplasmic signalling molecules include those that comprise, in order from the amino to carboxyl terminus, cytoplasmic signalling sequences derived from i) CD137, ii) TCRζ and iii) CD28; i) CD137, ii) CD28 and iii) TCRζ; i) TCRζ, ii) CD137 and iii) CD28; i) TCRζ, ii) CD28 and iii) CD137; i) CD28, ii) TCRζ and iii) CD137; and i) CD28, ii) CD 137 and iii) TCRζ.

The novel cytoplasmic signalling molecules of the invention can be used, either by themselves or, as a component part of a larger protein such as a chimeric receptor. As individual protein molecules, they can be introduced into, or expressed in, effector cells in order to act as substitute cytoplasmic signalling sequences for immune cell receptors already expressed within that cell. In this way they can increase the efficiency of signalling through the receptor. They may exist as soluble polypeptides in the cell cytoplasm, or they may be anchored or tethered to a cell membrane and extend into the cytoplasm, where they are capable of mediating signal transduction under a given set of physiological cellular conditions.

However, it is envisaged that the cytoplasmic signalling molecules of this invention are used preferentially to mediate signalling when employed as a cytoplasmic signalling domain of a chimeric receptor protein. Such chimeric receptors also comprise an extracellular ligand-binding domain and a transmembrane domain.

Thus, according this aspect of the invention there is provided a nucleic acid encoding a chimeric receptor protein comprising an extracellular ligand-binding domain, a transmembrane domain, and a cytoplasmic signalling domain wherein the cytoplasmic signalling domain mediates signal transduction through at least two different secondary messenger pathways.

The incorporation of an extracellular ligand-binding domain confers on the chimeric receptor the ability to exhibit specificity for a specific ligand or class of ligands. This specificity can be used to define precise ligands or classes of ligands that are capable of activating the receptor. In this way the receptor may be designed to activate the cell in which it is expressed upon binding a chosen class of, or individual, ligand.

Contact between the ligand and its corresponding binding domain in a chimeric receptor, results in signal transduction through the cytoplasmic signalling domain. The combination of cytoplasmic signalling sequences within the cytoplasmic signalling molecule of the invention chosen to act as a cytoplasmic domain of the chimeric receptor, dictates the magnitude of the signal transduced, and consequently controls the level to which the cell is activated.

A further embodiment of the invention thus provides nucleic acids encoding chimeric receptor proteins comprising an extracellular ligand-binding domain, a transmembrane domain, and a cytoplasmic signalling domain wherein the cytoplasmic signalling domain is encoded by a nucleic acid encoding a cytoplasmic signalling molecule according to any of the previously described aspects of the invention.

The term “extracellular ligand-binding domain” as used herein, is defined as any oligo- or polypeptide that is capable of binding a ligand. Accordingly antibody binding domains, antibody hypervariable loops or CDRs, receptor binding domains and other ligand binding domains, examples of which will be readily apparent to the skilled artisan, are described by this term. Preferably the domain will be capable of interacting with a cell surface molecule.

Examples of proteins associated with binding to cell surface molecules that are of particular use in this invention include, antibody variable domains (V_{H} or V_{L}), T-cell receptor variable region domains (TCRα, TCRβ, TCRγ, TCRδ), or the chains of CD8α, CD8b, CD11A CD11B, CD11C, CD18, CD29, CD49A, CD49B, CD49D, CD49E, CD49F, CD61, CD41, or CD51. Whilst it may be of benefit to use the entire domain or chain in some instances, fragments may be used where appropriate. Fab′ fragments or, especially single chain Fv fragments, are particularly useful binding components.

The choice of domain will depend upon the type and number of ligands that define the surface of a target cell. For example, the extracellular ligand binding domain may be chosen to recognise a ligand that acts as a cell surface marker on target cells associated with a particular disease state. Thus examples of cell surface markers that may act as ligands include those associated with viral, bacterial and parasitic infections, autoimmune disease and cancer cells. In the latter case, specific examples of cell surface markers are the bombesin receptor expressed on lung tumour cells, carcinoembryonic antigen (CEA), polymorphic epithelial mucin (PEM), CD33, the folate receptor, epithelial cell adhesion molecule (EPCAM) and erb-B2. Other ligands of choice are cell surface adhesion molecules, inflammatory cells present in autoimmune disease, and T cell receptors or antigens that give rise to autoimmunity. The potential ligands listed above are included by way of example; the list is not intended to be exclusive and further examples will be readily apparent to those of skill in the art.

Chimeric receptors may be designed to be bi- or multi-specific i.e. they may comprise more than one ligand binding domain and therefore, be capable of exhibiting specificity for more than one ligand. Such receptors may recruit cellular immune effector cells (e.g. T cells, B cells, natural killer (NK) cells, macrophages, neutrophils, eosinophils, basophils, or mast cells), or components of the complement cascade.

A further component of a chimeric receptor is the transmembrane domain. This may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the α, β or ζ chain of the T-cell receptor, CD28, CD3ε, CD45, CD4, CD5, CD8, CD9, CD16, CD22, CD33, CD37, CD64, CD80, CD86, CD134, CD137, CD154. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signalling domain of the chimeric receptor. A glycine-serine doublet provides a particularly suitable linker.

Between the extracellular ligand-binding domain and the transmembrane domain, or between the cytoplasmic signalling domain and the transmembrane domain, there may be incorporated a spacer domain. As used herein, the term “spacer domain” generally means any oligo- or polypeptide that functions to link the transmembrane domain to, either the extracellular ligand-binding domain or, the cytoplasmic signalling domain in the polypeptide chain. A spacer domain may comprise up to 300 amino acids, preferably 10 to 100 amino acids and most preferably 25 to 50 amino acids.

Spacer domains may be derived from all or part of naturally occurring molecules, such as from all or part of the extracellular region of CD8, CD4, or CD28, or from all or part of an antibody constant region. Alternatively, the spacer may be a synthetic sequence that corresponds to a naturally occurring spacer sequence, or may be an entirely synthetic spacer sequence.

Spacer domains may be designed in such a way that they, either minimise the constitutive association of chimeric receptors, thus reducing the incidence of constitutive activation in the cell or, promote such associations and enhance the level of constitutive activation in the cell. Either possibility may be achieved artificially by deleting, inserting, altering or otherwise modifying amino acids and naturally occurring sequences in the transmembrane and/or spacer domains, which have side chain residues that are capable of covalently or non-covalently interacting with the side chains of amino acids in other polypeptide chains. Particular examples of amino acids that can normally be predicted to promote association include cysteine residues, charged amino acids or amino acids such as serine or threonine within potential glycosylation sites.

Chimeric receptors may be designed in such a way that the spacer and transmembrane components have free thiol groups, thereby providing the receptor with multimerisation, and particularly dimerisation, capacity. Such multimeric receptors are preferred, especially dimers. Receptors with spacer domains derived from CD28 components and/or antibody hinge sequences and transmembrane regions derived from CD28 and the zeta chain of the natural T cell receptor are especially preferred.

The current invention not only provides the nucleic acids encoding novel cytoplasmic signalling molecules and chimeric receptor proteins, but also extends to the proteins themselves.

Nucleic acid coding sequences of the cytoplasmic signalling sequences for use in this invention, are readily derived from the specified amino acid sequences. Other nucleic acid sequences are widely reported in the scientific literature and are also available in public databases. DNA may be commercially available, may be part of cDNA libraries, or may be generated using standard molecular biology and/or chemistry procedures as will be clear to those of skill in the art. Particularly suitable techniques include the polymerase chain reaction (PCR), oligonucleotide-directed mutagenesis, oligonucleotide-directed synthesis techniques, enzymatic cleavage or enzymatic filling-in of gapped oligonucleotide. Such techniques are described by Sambrook & Fritsch 1989, and in the examples contained hereinafter.

The nucleic acids of the invention may be used with a carrier. The carrier may be a vector or other carrier suitable for the introduction of the nucleic acids ex-vivo or in-vivo into target cell and/or target host cells. Examples of suitable vector include viral vectors such as retroviruses, adenoviruses, adeno-associated viruses (AAVs), Epstein-Barr virus (EBV) and Herpes simplex virus (HSV). Non-viral vector may also be used, such as liposomal vectors and vectors based on condensing agents such as the cationic lipids described in International patent application numbers WO96/10038, WO97/18185, WO97/25329, WO97/30170 and WO97/31934. Where appropriate, the vector may additionally include promoter and regulatory sequences and/or replication functions from viruses, such as retrovirus long terminal repeats (LTRs), AAV repeats, SV40 and human cytomegalovirus (hCMV) promoters and/or enhancers, splicing and polyadenylation signals and EBV and BK virus replication functions. Tissue-specific regulatory sequences such as the TCR-α promoter, E-selectin promoter and the CD2 promoter and locus control region may also be used. The carrier may be an antibody.

The invention also includes cloning and expression vectors containing a nucleic acid according to any of the above-described aspects of the invention. Such expression vectors will incorporate the appropriate transcriptional and translation control sequences, for example, enhancer elements, promoter-operator regions, termination stop sequence, mRNA stability sequences, start and stop codons or ribosome binding sites, linked where appropriate in-frame with the nucleic acid molecules of the invention.

Additionally in the absence of a naturally effective signal peptide in the protein sequence, it may be convenient to cause recombinant cytoplasmic signalling proteins to be secreted from certain hosts. Accordingly, further components of such vectors may include nucleic acid sequences encoding secretion signalling and processing sequences.

Vectors according to the invention include plasmids and viruses (including both bacteriophage and eukaryotic viruses). Many expression systems suitable for the expression of heterologous proteins are well known and documented in the art. For example, the use of prokaryotic cells such as Escherichia coli to express heterologous polypeptides and polypeptide fragments is well established (see for example, Sambrook & Frtsch, 1989, Glover, 1995a). Similarly, eukaryotic expression systems have been well developed and are commonly used for heterologous protein expression (see for example, Glover, 1995b and O'Reilly et al., 1993). In eukaryotic cells, apart from yeasts, the vectors of choice are virus-based. Particularly suitable viral vectors include baculovirus-, adenovirus-, and vaccinia virus-based vectors.

Vectors containing the relevant regulatory sequences (including promoter, termination, polyadenylation, and enhancer sequences, marker genes) can either be chosen from those documented in the literature, or readily constructed for the expression of the proteins of this invention using standard molecular biology techniques. Such techniques, and protocols for the manipulation of nucleic acids, for example in the preparation of nucleic acid constructs, mutagenesis, sequencing, DNA transformation and gene expression, as well as the analysis of proteins, are described in detail in Ausubel et al, 1992 or Rees et al., 1993.

Suitable host cells for the in vitro expression of the cytoplasmic signalling molecules and chimeric receptor proteins of the invention include prokaryotic cells e.g. E. coli, eukaryotic yeasts e.g Saccharomyces cerevisiae, Pichia species, Schizosaccharomyces pombe, mammalian cell lines and insect cells. Alternatively chimeric receptors of the invention may be expressed in vivo in a variety of host such as, for example, insect larvae, plant cells, or more preferably mammalian tissues.

Nucleic acid may be introduced into a host cell by any suitable technique. In eukaryotic cells these techniques may include calcium phosphate transfection, DEAE-Dextran, electroporation, particle bombardment, liposome-mediated transfection or transduction using retrovirus, adenovirus or other viruses, such as vaccinia or, for insect cells, baculovirus. In bacterial cells, suitable techniques may include calcium chloride transformation, electroporation or transfection using bacteriophage. The nucleic acid may remain in an episomal form within the cell, or it may integrate into the genome of the cell. If the latter is desired, sequences that promote recombination with the genome will be included in the nucleic acid. Following introduction of the nucleic acid into host cells, the cells may be cultured under conditions to enhance or induce expression of the chimeric receptor protein as appropriate.

Thus, further aspects of the invention provide host cells containing a nucleic acid encoding a cytoplasmic signalling molecule and/or chimeric receptor protein as described herein, and host cells expressing such proteins.

According to still further aspects, the nucleic acids of the invention can be employed in either ex-vivo or in-vivo therapies.

For ex-vivo use the nucleic acid may be introduced into effector cells (removed from the target host) using methods well known in the art e.g. transfection, transduction (including viral transduction), biolistics, protoplast fusion, calcium phosphate mediated DNA transformation, electroporation, cationic lipofection, or targeted liposomes. The effector cells are then reintroduced into the host using standard techniques. Examples of suitable effector cells for the expression of the adaptor receptors of the present invention include cells associated with the immune system such as lymphocytes e.g. cytotoxic T-lymphocytes, tumour infiltrating lymphocytes, neutrophils, basophils, or T-helper cells, dendritic cells, B-cells, haematopoietic stem cells, macrophages, monocytes or NK cells. The use of cytotoxic T-lymphocytes is especially preferred.

Nucleic acids of the invention are particularly suitable for in vivo administration. In order to achieve this, the nucleic acid, preferably DNA, may be in the form of a targeted carrier system in which a carrier as described above is capable of directing the nucleic acid to a desired effector cell. Examples of suitable targeted delivery systems include targeted naked DNA, targeted liposomes encapsulating and/or complexed with the DNA, targeted retroviral systems and targeted condensed DNA such as protamine and polylysine-condensed DNA.

Targeting systems are well known in the art and include, for example, using antibodies or fragments thereof against cell surface antigens expressed on target cells in vivo such as CD8, CD16, CD4, CD3, selecting (e.g. E-selectin), CD5, CD7, CD24, and activation antigens (e.g. CD69 an dIL-2R. Alternatively other receptor-ligand interactions can be used for targeting e.g. CD4 to target HIV_{gp}160-expressing target cells.

In general, the use of antibody-targeted DNA is preferred, particularly antibody-targeted naked DNA, antibody-targeted condensed DNA and especially antibody-targeted liposomes. Types of liposomes that may be used include for example pH-sensitive liposomes, where linkers that are cleaved at low pH may be used to link the antibody to the liposome. The nucleic acids of the present invention may also be targeted directly to the cytoplasm by using cationic liposomes, which fuse with the cell membrane. Liposomes for use in the invention may also have hydrophilic molecules, e.g. polyethylene glycol polymers, attached to their surface to increase their circulating half-life. There are many example in the art of suitable groups for attaching DNA to liposomes or other carriers; see for example International patent application numbers WO88/04924, WO90/09782, WO91/05545, WO91/05546, WO93/19738, WO94/20073 and WO94/22429. The antibody or other targetting molecule may be linked to the DNA, condensed DNA or liposome using conventional linking groups and reactive functional groups in the antibody, e.g. thiols or amines, and in the DNA or DNA-containing material.

Non-targeted carrier systems may also be used. In these systems targeted expression of the protein is advantageous. This may be achieved, for example, by using T cell specific promoter systems such as the zeta promoter, CD2 promoter and locus control region, CD4, CD8 TCRα and TCRβ promoters, cytokine promoters, such as the IL2 promoter, and the perforin promoter.

It is intended that the cytoplasmic signalling molecules and chimeric receptor proteins of the present invention, or the nucleic acids encoding them, be applied in methods of therapy of mammalian, particularly human, patients. Cytoplasmic signalling molecules and chimeric receptors generated by the present invention may be particularly useful in the treatment of a number of diseases or disorders. Such diseases or disorders may include those described under the general headings of infectious diseases, e.g. HIV infection; inflammatory disease/autoimmunity e.g. asthma, eczema; congenital e.g. cystic fibrosis, sickle cell anaemia; dermatologic, e.g. psoriasis; neurologic, e.g. multiple sclerosis; transplants e.g. organ transplant rejection, graft-versus-host disease; metabolic/idiopathic disease, e.g. diabetes; cancer.

For example, expression of a chimeric receptor of the invention on the surface of a T cell may initiate the activation of that cell upon binding of the ligand-binding domain to a ligand on a target cell. The ensuing release of inflammatory mediators stimulated by the activation of the signalling function of the receptor ensures destruction of the target cell.

When a chimeric receptor according to the present invention is expressed in an effector cell of the immune system, binding to target will activate the effector cell; downstream effects of this activation may also result in the destruction of the target cell. If the extracellular ligand-binding domain of the chimeric receptor exhibits specificity for a surface marker on an immune cell, effector cells may be recruited to the site of disease. Accordingly, expression of a chimeric receptor of the invention in a diseased cell will ensure its destruction.

The expression of multispecific chimeric receptor proteins, or more than one chimeric receptor (with different ligand specificities), within a single host cell, may confer dual functionality on the receptor. For example, binding of the chimeric receptor to its target may not only activate the effector cell itself, but may additionally attract other immune effectors to the site of disease. The target cell may thus be destroyed by the activation of the immune system.

A further aspect of the invention provides a composition comprising a cytoplasmic signalling molecule, or a chimeric receptor protein, or a nucleic acid(s) encoding a cytoplasmic signalling molecule or chimeric receptor protein, according to any of the aspects of the invention described above, in conjunction with a pharmaceutically acceptable excipient.

Suitable excipients will be well known to those of skill in the art and may, for example, comprise a phosphate-buffered saline (e.g. 0.01M phosphate salts, 0.138M NaCl, 0.0027M KCl, pH7.4), a liquid such as water, saline, glycerol or ethanol, optionally also containing mineral acid salts such as hydrochlorides, hydrobromides, phosphates, sulphates and the like; and the salts of organic acids such as acetates propionates, malonates, benzoates and the like. Auxiliary substances such as wetting or emulsifying agents, and pH buffering substances, may also be present. A thorough discussion of pharmaceutically acceptable excipients is available in Remington's Pharmaceutical Sciences (Mack Pub. Co., N.J. 1991). Preferably, the compositions will be in a form suitable for parenteral administration e.g. by injection or infusion, for example by bolus injection or continuous infusion or particle-mediated injection. Where the composition is for injection or infusion, it may take the form of a suspension, solution or emulsion in an oily or aqueous vehicle and it may contain formulatory agents such as suspending, preservative, stabilising and/or dispersing agents. Alternatively, the composition may be in dry form, for reconstitution before use with an appropriate sterile liquid. For particle-mediated administration, DNA may be coated on particles such as microscopic gold particles.

A carrier may also be used that does not itself induce the production of antibodies harmful to the individual receiving the composition and which may be administered without undue toxicity. Suitable carriers are typically large, slowly metabolised macromolecules such as proteins, polysaccharides, polylactic acids, polyglycolic acids, polymeric amino acids, amino acid copolymers and inactive virus particles. Pharmaceutical compositions may also contain preservatives in order to prolong shelf life in storage.

If the composition is suitable for oral administration, the formulation may contain, in addition to the active ingredient additives such as starch (e.g. potato, maize or wheat starch, cellulose), starch derivatives such as microcrystalline cellulose, silica, various sugars such as lactose, magnesium carbonate and/or calcium phosphate. It is desirable that a formulation suitable for oral administration be well tolerated by the patient's digestive system. To this end, it may be desirable to include mucus formers and resins. It may also be desirable to improve tolerance by formulating the compositions in a capsule that is insoluble in the gastric juices. In addition, it may be preferable to include the composition in a controlled release formulation.

According to yet a further aspect of the invention the use of the nucleic acids encoding novel cytoplasmic signalling molecules or chimeric receptor proteins, or of the polypeptides so encoded, or of a pharmaceutical composition containing such nucleic acids or polypeptides, in the manufacture of a medicament for the treatment or prevention of disease in humans or in animals is also provided.

The various aspects and embodiments of the present invention will now be illustrated in more detail by way of example. It will be appreciated that modification of detail may be made without departing from the scope of the invention.

### BRIEF DESCRIPTION OF THE FIGURES

FIG. 1: Cloning cassette for construction of chimeric receptors comprising cytoplasmic signalling domains containing a CD137-derived cytoplasmic signalling sequence.

FIG. 2: Nucleotide and amino acid sequence of h.CD28 extracellular spacer and the human CD28 transmembrane region used in the construction of the cloning cassette describe in FIG. 1.

FIG. 3: Oligonucleotide sequences used in the construction of cytoplasmic signalling molecules comprising a CD137-derived cytoplasmic signalling sequence.

FIG. 4: Antigen specific stimulation of chimeric receptor proteins having cytoplasmic signalling domains comprising a cytoplasmic signalling sequence derived from CD137—stimulation with cell-bound antigen.

FIG. 5: Antigen specific stimulation of chimeric receptor proteins having cytoplasmic signalling domains comprising a cytoplasmic signalling sequence derived from CD137—stimulation with soluble antigen.

### EXAMPLES

### Example 1

### Construction of Receptor Cloning Cassette

To facilitate construction and analysis of signalling region combinations in a chimeric receptor format, a cloning cassette was devised in pBluescript SK+ (Stratagene) and pcDNA3 (Invitrogen). This cloning cassette consists of a binding component cassette and a spacer/transmembrane cassette.

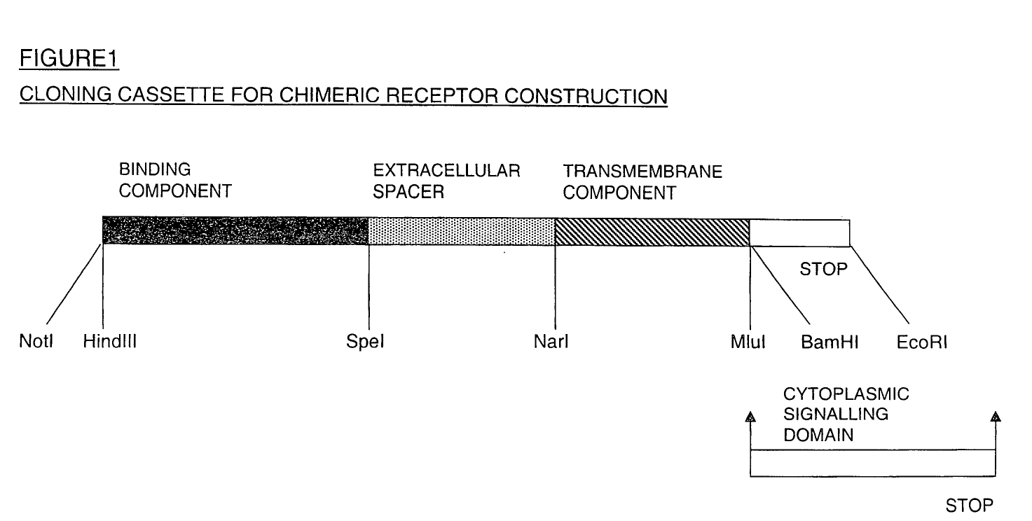

This new cassette system is shown in FIG. 1. The binding component has 5′ (relative to coding direction) Not I and Hind III restriction sites and a 3′ (again relative to coding direction) Spe I restriction site. The extracellular spacer is flanked by a Spe I site (therefore encoding Thr, Ser at the 5′ end) and a Nar I site (therefore encoding Gly, Ala at the 3′ end). The transmembrane component is flanked by a Nar I site at its 5′end (therefore encoding Gly, Ala) and by Mlu I (therefore encoding Thr, Arg) and BamH I sites (therefore encoding Gly, Ser) at the 3′ end. The cytoplasmic signalling domain may be cloned in-frame into the BamH I site. Following this BamH I site there is a stop codon for transcription termination and there is also an EcoR I site situated downstream of this to facilitate the subsequent rescue of whole constructs.

a) Binding Component Cassette (Hind III to Spe I) This consists of the V_{L} and V_{H} regions of an anti-CD33 antibody joined by a short linker sequence. The V_{L} region was PCR cloned from hP67scFv (WO97/23613) with oligos A5267 and F22785 and digested with restriction enzymes to generate a Hind III to Bgl II fragment. The V_{H} region was PCR cloned from hP67scFv (WO97/23613) with oligos F22786 and F22787 and digested with restriction enzymes to generate a Bgl II to SpeI fragment. These two fragments were then co-ligated together with the linker sequence into a cloning vector. The linker was formed by annealing oligos F22966 and F22967 which have 5′ phosphate groups: these annealed oligos were compatible with Bgl II overhangs but did not reform the Bgl II site on ligation.

b) Spacer/Transmembrane Cassette (Spe I to EcoRI with internal BamHI site for cloning signalling cassettes prior to a stop codon) The extracellular spacer component h.CD28, consists of residues 234 to 243 of human IgG1 hinge and residues 118 to 134 of human CD28. The transmembrane component consists of residues 135 to 161 of human CD28 (Aruffo & Seed 1987).

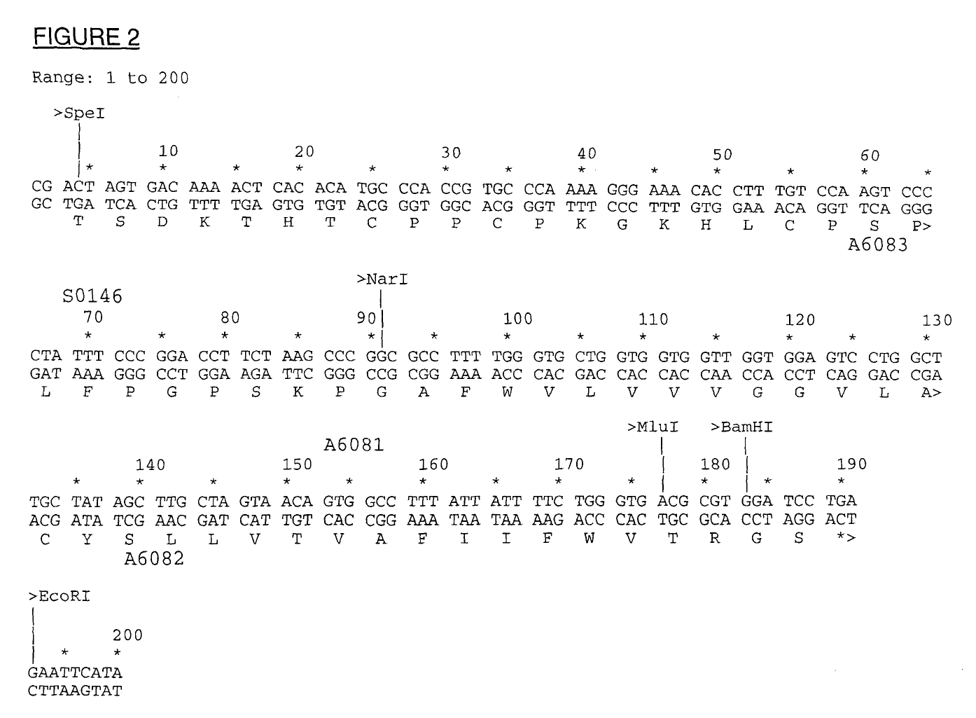

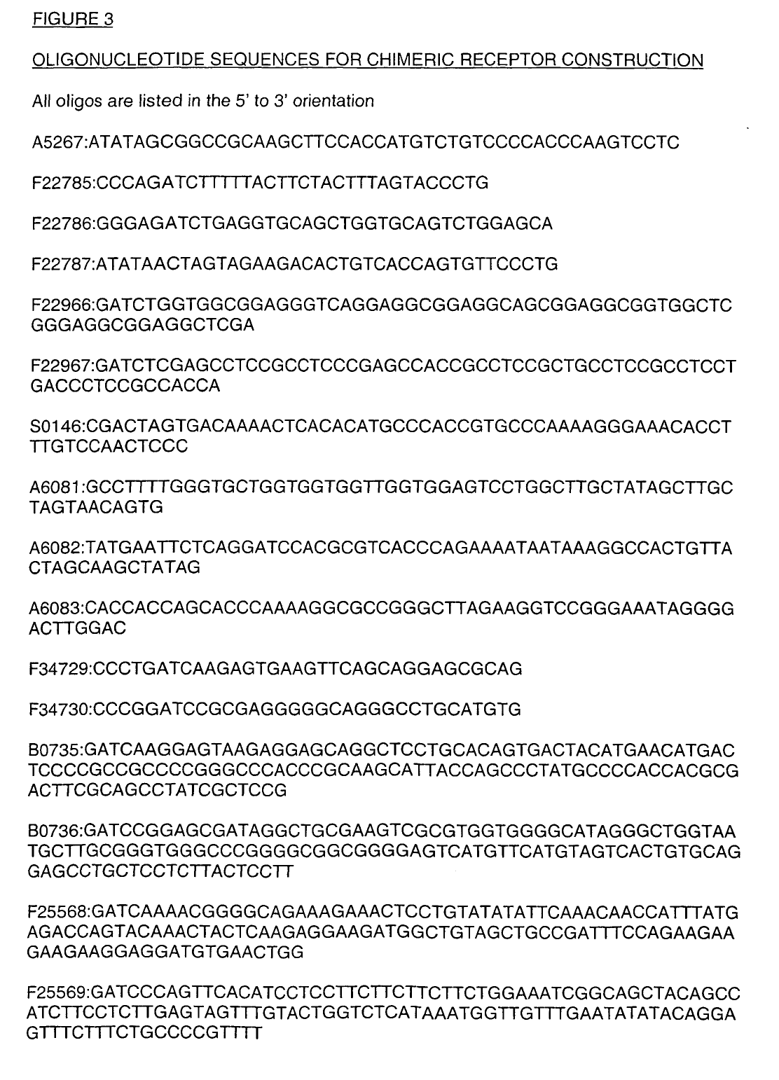

To generate this cassette, a 200 bp fragment was PCR assembled using oligos: S0146, A6081, A6082 and A6083 (FIG. 3). This fragment starts with a SpeI site and consists of the extracellular spacer h.CD28, the human CD28 transmembrane region, a BamHI site, a stop codon and finishes with an EcoRI site (see FIG. 2).

### Example 2

### Construction of Chimeric Receptors with Different Combinations of Cytoplasmic Signalling Sequences

Cytoplasmic signalling sequences were then cloned into the BamHI site of the above cassette. Because each signalling sequence is on a BcII to BamHI fragment, directional cloning of these sequences retains a BamHI site at the 3′ end only, allowing subsequent cloning of a second and third cytoplasmic signalling sequence. This cloning method generates a 2 amino acid spacer (Gly,Ser) between each signalling sequence.

a) Zeta Signalling Cassette (Bcl I to BamH I fragment) This consists of residues 31 to 142 of human TCRζ chain (Weissman et al 1988, Moingeon et al., 1990) and was PCR cloned using oligos F34729 and F34730 from pHMF492 (a zeta chimeric receptor described in International Patent application PCT/GB00/01456) and digested with restriction enzymes BcII and BamHI.

b) CD28 Signalling Cassette (Bcl I to BamH I fragment) This comprises the intracellular component of human CD28 of consists residues 162 to 202 of CD28 (Aruffo & Seed 1987). This component was formed by annealing oligos with 5′ phosphate groups B0735 and B0736: the single stranded overhangs of these annealed oligos form a 5′ BcII half site and a 3′ BamHI half site.

c) CD137 Signalling Cassette (Bc I to BamH I fragment) This consists of residues 214 to 255 comprising the intracellular component of human CD137 (Alderson et al., 1994) and was formed by annealing oligos with 5′ phosphate groups F25568 and F25569: the single stranded overhangs of these annealed oligos form a 5′ BcII half site and a 3′ BamHI half site. Chimeric receptors generated from the above-described signalling cassettes are listed in Table 1.

### Example 3

### Analysis of Receptors

a) Construction of expression plasmids. The chimeric receptor constructs were sub-cloned from pBluescript KS+ into the expression vector pEE6hCMV.ne (Cockett, et al., 1991) on a Hind III to EcoR I restriction fragment. The empty expression vector (i.e the base vector lacking in chimeric receptor genes) is used as a negative control.

TABLE 1
Components of chimeric receptors constructed with various cytoplasmic signalling domains. Chimeric
receptors are referred to in the examples herein according to the construction of their cytoplasmic signalling
domain. Thus a “zeta” chimeric receptor refers to the first chimeric receptor described in the table below,
and a “CD28-zeta-CD137” refers to the seventh chimeric receptor described below.

| | | | Cytoplasmic Signalling Domain | | | | |
|---|---|---|---|---|---|---|---|
| | | | (formed by cytoplasmic signalling molecule comprising the | | | | |
| | | | components described below) | | | | |
| | | | Cytoplasmic | | Cytoplasmic | | Cytoplasmic |
| Ligand Binding | | Transmembrane | Signalling | | Signalling | | Signalling |
| Domain | Spacer | Domain | Sequence #1 | Spacer | Sequence #2 | Spacer | Sequence #3 |
| hP67 scFv | h.CD28 | CD28 | TCRζ | — | — | — | — |
| hP67 scFv | h.CD28 | CD28 | CD137 | — | — | — | — |
| hP67 scFv | h.CD28 | CD28 | CD28 | — | — | — | — |
| hP67 scFv | h.CD28 | CD28 | CD137 | Gly-Ser | CD28 | — | — |
| hP67 scFv | h.CD28 | CD28 | CD28 | Gly-Ser | CD137 | — | — |
| hP67 scFv | h.CD28 | CD28 | TCRζ | Gly-Ser | CD137 | — | — |
| hP67 scFv | h.CD28 | CD28 | CD137 | Gly-Ser | TCRζ | — | — |
| hP67 scFv | h.CD28 | CD28 | CD28 | Gly-Ser | CD137 | Gly-Ser | TCRζ |
| hP67 scFv | h.CD28 | CD28 | TCRζ | Gly-Ser | CD137 | Gly-Ser | CD28 |
| hP67 scFv | h.CD28 | CD28 | CD137 | Gly-Ser | TCRζ | Gly-Ser | CD28 |
| hP67 scFv | h.CD28 | CD28 | CD28 | Gly-Ser | TCRζ | Gly-Ser | CD137 |
| hP67 scFv | h.CD28 | CD28 | CD137 | Gly-Ser | CD28 | Gly-Ser | TCRζ |
| hP67 scFv | h.CD28 | CD28 | TCRζ | Gly-Ser | CD28 | Gly-Ser | CD137 |

b) Transfection into Jurkat E6.1 cells. To generate stable cell lines, the expression plasmids were linearised and transfected into Jurkat E6.1 cells (ECACC) by electroporation using a BioRad Gene Pulser. Cells (˜2.5×10⁶) were mixed with DNA (10 μg) and pulsed twice at 1 kV, 3 μF (0.4 cm electrode gap cuvette) in 1 ml PBS. The cells were left to recover overnight in non-selective media before being selected and cultured in media supplemented with the antibiotic G418 (Sigma) at 1.5 mg/ml. After approximately four weeks cells were ready for analysis.

c) Analysis of surface expression: FACS. Approximately 5×10⁵ Jurkat cells were stained with 1 μg/ml FITC labelled CD33 antigen. Fluorescence was analysed by a FACScan cytometer (Becton Dickinson).

d) Analysis of function: IL-2 production. 2×10⁵ cells were incubated at 37° C. in 8% CO₂ for 20 hours in 96 well plates with target cells at an effector:target ratio of 1:1 and 1:2 or soluble CD33 antigen at a concentration of 300 ng/ml and 100 ng/ml. Cell supernatants were then harvested and assayed for human IL-2 (R & D Systems Duoset ELISA development kit).

The target cells used were:

N.EE6—a mouse myeloma (NS0) transfected with a control expression vector. These cells are used as a negative control target cell line.

N.CD33—a mouse myeloma (NS0) transfected with an expression vector facilitating the expression of antigen CD33 on the cell surface.

### Example 4

### Results

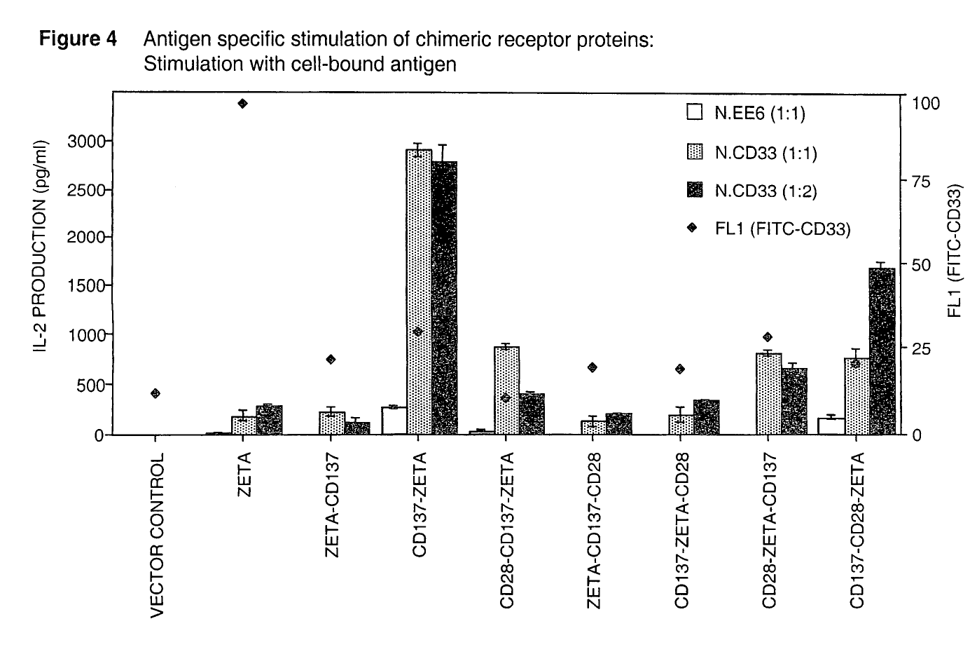

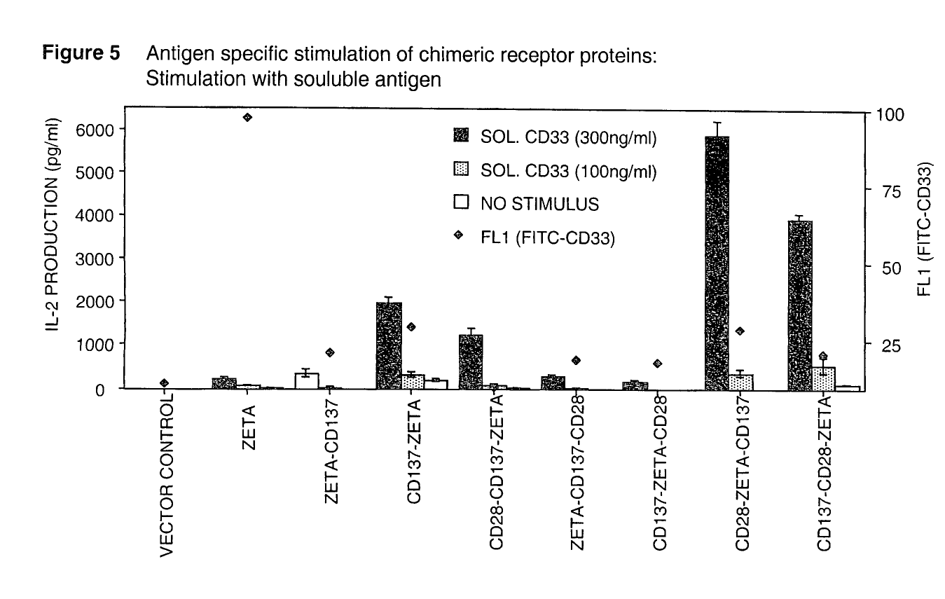

FIGS. 4 and 5 show that when a cytoplasmic signalling sequence from CD137 is incorporated in a cytoplasmic signalling molecule, which is employed as a cytoplasmic signalling domain of a chimeric receptor, binding of ligand to the extracellular ligand binding domain results in cellular activation, as indicated by the production of IL-2. When corrected for the level of expression of receptors using the FITC-CD33 data, it can be seen that chimeric receptors comprising signalling domains having a cytoplasmic signalling sequence derived from CD137 in conjunction with at least one additional cytoplasmic signalling sequence are more efficient at mediating IL-2 production than traditional chimeric receptors such as the zeta chimeric receptor.

### REFERENCES CITED

Aruffo, A & Seed, B. 1987 Proceedings of the National Academy of Sciences USA 84: 8573-8577.

Alderson, M. R., Smith, C. A., Tough, T. W., Davis-Smith, T., Armitage R. J., Falk, B., Roux, E., Baker, E., Sutherland, G. R. & Din, W. S. (1994) European Journal of Immunology 24:2219-27

Ausubel, F. W. & Kingston R. E. (1992) Short protocols in Molecular Biology. 2nd edition. John Wiley & Sons Ltd.

Cockett, M. I., Bebbington, C. R. & Yarranton, G. T. (1991) Nucleic Acids Research 19:319-325.

DeBenedette, M. A., Chu, N. R., Pollok, K. E., Hurtado, J., Wade, W. F., Kwon, B. S. & Watts, T. H. (1995) Journal of Experimental Medicine 181:985-992.

DeBenedette, M. A., Shahinian, A., Make, T. W. & Watts, T. H. (1997) Journal of Immunology 158:551-559.

DeFranco A. L. (1997) Current Opinion in Immunology 9:296-308.

Finney, H. M., Lawson, A. D. G., Bebbington, C. R. & Weir, A. C. N. (1998) Journal of Immunology 161:2791-2797.

Glover, D. M. 1995a. DNA cloning: a practical approach, Volume II: Expression systems. IRL press.

Glover, D. M. 1995b. DNA cloning: a practical approach, Volume IV: Mammalian systems. IRL press.

Goodwin, R. G., Dins, W. S., Davis-Smith, T., Anderson, D. M., Gimpel, S. D., Sato, T., Maliszewski, A. C., Brannan, R. C. I., Copeland, N. G., Jenkins, N. A. et al. (1993) European Journal of Immunology 23:2631-2641.

Kuwana, Y., Asakura, Y., Utsunomiya, N., Nakanishi, M., Arat, Y., Itoh, S., Nagese, F. & Kurosawa, Y. (1987) Biochemical and Biophysical Research Communications 149:960-968.

Kwon, B. S. & Weissman, S. M. (1989) Proceedings of the National Acadamey of Sciences USA 86:1963-1967.

Kwon, B. Moon, C. H., Kang, S., Seo, S. K. & Kwon, B. S. (2000) Molecules and Cells 10:119-126.

Moingeon, P., Stebbins, C. C., D'Adamio, L., Lucich, J. & Reinherz, E. L. (1990) European Journal of Immunology 20:1741-1745.

O'Reilly, D. R., & Miller, L. K. (1993). Baculovirus expression vectors—A laboratory manual. Oxford University Press.

Rees, A. R (1993) Protein Engineering: a practical approach. IRL press

Reth, M. (1989) Nature, 338:383-384

Romeo, C., Kolanus, W., Amiot, M. & Seed, B. (1992) Cold Spring Harbour Symposia on Quantitative Biology. Volume LVII pp 117-125.

Sambrook, J., & Fritsch, E. (1989) Molecular cloning: a laboratory manual. 2nd edition. Cold Spring Harbour Press, N.Y.

Sancez-Garcia, F. J., Aller, W. W. & McCormack, W. T. (1997) Immunology 91:81-87.

Weiss, A. & Littman, D. R. (1994) Cell 76:26-274.

(Weissman et al 1988, PNAS 85,9709-9713,1988

1. A nucleic acid encoding a cytoplasmic signalling molecule comprising at least two cytoplasmic signalling sequences, wherein at least one cytoplasmic signalling sequence is derived from CD137.

2. A nucleic acid according to claim 1, wherein at least one cytoplasmic signalling sequence is a primary cytoplasmic signalling sequence.

3. A nucleic acid according to claim 2 wherein the primary signalling sequence contains an ITAM.

4. A nucleic acid according to claim 3, wherein the primary signalling sequence is derived from TCRζ, FcRγ, FcRβ, CD3γ, CD3δ, CD3ε, CD5, CD22, CD79a, CD79b or CD66d.

5. A nucleic acid according to claim 3, wherein the primary signalling sequence contains an ITIM.

6. A nucleic acid according to claim 1, wherein at least one cytoplasmic signalling sequence is a secondary cytoplasmic signalling sequence.

7. A nucleic acid according to claim 6, wherein the secondary cytoplasmic signalling sequence is derived from CD2, CD4, CD8, CD28, CD134 or CD154.

8. A nucleic acid according to any one of claims 2 to 7, which encodes three cytoplasmic signalling sequences.

9. A nucleic acid according to any one of claims 2 to 7, wherein the first cytoplasmic signalling sequence encoded for in reading frame is derived from CD137.

10. A nucleic acid according to claim 9, which encodes i) a cytoplasmic signalling sequence derived from CD137 followed in reading frame by ii) a cytoplasmic signalling sequence derived from TCRζ.

11. A nucleic acid according to any one of claims 2 to 7, wherein the second cytoplasmic signalling sequence encoded for in reading frame is derived from CD137.

12. A nucleic acid according to claim 11, which encodes i) a cytoplasmic signalling domain derived from TCRζ followed in reading frame by ii) a cytoplasmic signalling domain derived from CD137.

13. A nucleic acid according to claim 8, wherein the first cytoplasmic signalling sequence encoded for in reading frame is derived from CD137 or from a secondary cytoplasmic signalling sequence.

14. A nucleic acid encoding to claim 13 which encodes in reading frame i) a cytoplasmic signalling sequence derived from CD28, ii) a cytoplasmic signalling sequence derived from CD137, and iii) a cytoplasmic signalling domain derived from TCRζ.

15. A nucleic acid encoding a chimeric receptor protein, which comprises an extracellular ligand-binding domain, a transmembrane domain and a cytoplasmic signalling domain, wherein the cytoplasmic signalling domain is encoded by a nucleic acid according to any one of claims 1 to 14.

16. A nucleic acid according to claim 15, wherein the extracellular ligand-binding domain is an antibody, or an antigen-binding fragment thereof.

17. A nucleic acid according to claim 16 wherein the antigen binding fragment is a Fab′ or scFv.

18. A nucleic acid according to any one of claims 15 to 17, wherein the transmembrane domain is derived from the α, β or ζ chain of the T-cell receptor, CD28, CD3ε, CD45, CD4, CD5, CD8, CD9, CD16, CD22, CD33, CD37, CD64, CD80, CD86, CD134, CD137, or CD154.

19. A nucleic acid according to claim 18 wherein the transmembrane domain is derived from CD28.

20. A vector comprising a nucleic acid according to any one of the preceding claims.

21. A host cell containing a nucleic acid according to any one of claims 1 to 19, or a vector according to claim 20.

22. A peptide or polypeptide comprising a cytoplasmic signalling molecule encoded by a nucleic acid according to any one of claims 1 to 14.

23. A chimeric receptor protein encoded by a nucleic acid according to any one of claims 15 to 19.

24. A host cell expressing a peptide or polypeptide according to claim 22 or a chimeric receptor protein, according to claim 23.

25. A nucleic acid according to any one of claims 1 to 19, or a vector according to claim 20, for use in therapy.

26. A chimeric receptor protein according to claim 23, for use in therapy.

27. A composition comprising a peptide or polypeptide according to claim 22, a chimeric receptor protein according to claim 23, a nucleic acid according to any one of claims 1 to 19, or a vector according to claim 20, in conjunction with a pharmaceutically acceptable excipient.

28. The use of a peptide or polypeptide according to claim 22, a chimeric receptor protein according to claim 23, or a composition according to claim 27, in the manufacture of a medicament for the treatment or prevention of disease in humans or in animals.


SEQUENCE LISTING (19 sequences)

SEQ ID NO: 1 - DNA, 200 bp, Artificial Sequence
  Synthetic Construct
cgactagtgacaaaactcacacatgcccaccgtgcccaaaagggaaacacctttgtccaagtcccctatttcccggaccttctaagcccggcgccttttgggtgctggtggtggttggtggagtcctggcttgctatagcttgctagtaacagtggcctttattattttctgggtgacgcgtggatcctgagaattcata

SEQ ID NO: 2 - DNA, 200 bp, Artificial Sequence
  Synthetic Construct
gctgatcactgttttgagtgtgtacgggtggcacgggttttccctttgtggaaacaggttcaggggataaagggcctggaagattcgggccgcggaaaacccacgaccaccaccaaccacctcaggaccgaacgatatcgaacgatcattgtcaccggaaataataaaagacccactgcgcacctaggactcctaagtat

SEQ ID NO: 3 - protein, 62 aa, Artificial Sequence
  Synthetic Construct
TSDKTHTCPPCPKGKHLCPSPLFPGPSKPGAFWVLVVVGGVLACYSLLVTVAFIIFWVTRGS

SEQ ID NO: 4 - DNA, 48 bp, Artificial Sequence
  Synthetic Construct
atatagcggccgcaagcttccaccatgtctgtccccacccaagtcctc

SEQ ID NO: 5 - DNA, 33 bp, Artificial Sequence
  Synthetic Construct
cccagatctttttacttctactttagtaccctg

SEQ ID NO: 6 - DNA, 36 bp, Artificial Sequence
  Synthetic Construct
gggagatctgaggtgcagctggtgcagtctggagca

SEQ ID NO: 7 - DNA, 38 bp, Artificial Sequence
  Synthetic Construct
atataactagtagaagacactgtcaccagtgttccctg

SEQ ID NO: 8 - DNA, 66 bp, Artificial Sequence
  Synthetic Construct
gatctggtggcggagggtcaggaggcggaggcagcggaggcggtggctcgggaggcggaggctcga

SEQ ID NO: 9 - DNA, 66 bp, Artificial Sequence
  Synthetic Construct
gatctcgagcctccgcctcccgagccaccgcctccgctgcctccgcctcctgaccctccgccacca

SEQ ID NO: 10 - DNA, 65 bp, Artificial Sequence
  Synthetic Construct
cgactagtgacaaaactcacacatgcccaccgtgcccaaaagggaaacacctttgtccaactccc

SEQ ID NO: 11 - DNA, 63 bp, Artificial Sequence
  Synthetic Construct
gccttttgggtgctggtggtggttggtggagtcctggcttgctatagcttgctagtaacagtg

SEQ ID NO: 12 - DNA, 67 bp, Artificial Sequence
  Synthetic Construct
tatgaattctcaggatccacgcgtcacccagaaaataataaaggccactgttactagcaagctatag

SEQ ID NO: 13 - DNA, 59 bp, Artificial Sequence
  Synthetic Construct
caccaccagcacccaaaaggcgccgggcttagaaggtccgggaaataggggacttggac

SEQ ID NO: 14 - DNA, 34 bp, Artificial Sequence
  Synthetic Construct
ccctgatcaagagtgaagttcagcaggagcgcag

SEQ ID NO: 15 - DNA, 33 bp, Artificial Sequence
  Synthetic Construct
cccggatccgcgagggggcagggcctgcatgtg

SEQ ID NO: 16 - DNA, 129 bp, Artificial Sequence
  Synthetic Construct
gatcaaggagtaagaggagcaggctcctgcacagtgactacatgaacatgactccccgccgccccgggcccacccgcaagcattaccagccctatgccccaccacgcgacttcgcagcctatcgctccg

SEQ ID NO: 17 - DNA, 129 bp, Artificial Sequence
  Synthetic Construct
gatccggagcgataggctgcgaagtcgcgtggtggggcatagggctggtaatgcttgcgggtgggcccggggcggcggggagtcatgttcatgtagtcactgtgcaggagcctgctcctcttactcctt

SEQ ID NO: 18 - DNA, 131 bp, Artificial Sequence
  Synthetic Construct
gatcaaaacggggcagaaagaaactcctgtatatattcaaacaaccatttatgagaccagtacaactactcaagaggaagatggctgtagctgccgatttccagaagaagaagaaggaggatgtgaactgg

SEQ ID NO: 19 - DNA, 133 bp, Artificial Sequence
  Synthetic Construct
gatcccaggttcacatcctccttcttcttcttctggaaatcggcagctacagccatcttcctcttgagtagtttgtactggtctcataaatggttgtttgaatatatacaggagtttctttctgccccgtttt